(19) United States
(12) Patent Application Publication
(10) Pub. No.: US 2021/0128431 A1
(43) Pub. Date: May 6, 2021

(54) NOURISHING AND CREAMY SHOWER CREAM

(71) Applicant: Henkel AG & Co. KGaA, Duesseldorf (DE)

(72) Inventors: KIRSTEN SARTINGEN, Schwalmtal (DE); BARBARA HEIDE, Krefeld (DE)

(73) Assignee: Henkel AG & Co. KGaA, Duesseldorf (DE)

(21) Appl. No.: 17/086,066

(22) Filed: Oct. 30, 2020

(30) Foreign Application Priority Data
Oct. 30, 2019 (DE) 10 2019 216 741.1

(51) Int. Cl. A61K 8/37; A61Q 5/02; A61K 8/73; A61K 8/92; A61Q 19/10

(52) U.S. Cl. CPC A61K 8/375; A61Q 5/02; A61Q 19/10; A61K 8/731; A61K 8/922; A61K 8/732

(57) ABSTRACT

A cosmetic cleansing product is described comprising (based on the total weight of the cleansing product)
a) from about 5 to about 18% by weight of at least one surfactant,
b) from about 1.5 to about 6% by weight of at least one starch and/or at least one hydrophobically modified starch compound,
c) from about 0.0005 to about 1% by weight of cellulose and/or a non-ionic cellulose ether,
d) from about 0.01 to about 5% by weight of at least one vegetable oil and/or vegetable butter.

### CROSS-REFERENCE TO RELATED APPLICATION

This application claims priority to German Patent Application No. 10 2019 216 741.1, filed Oct. 30, 2019, which is incorporated herein by reference in its entirety.

### TECHNICAL FIELD

The present disclosure is in the field of cosmetics and concerns cosmetic cleansing products comprising a specific combination of surfactants, starch (derivatives), cellulose (derivatives) and vegetable oils and/or butters.

### BACKGROUND

Cosmetic detergents have been known for a long time and are continuously improved or adapted to the changing needs of consumers.

Modern cleaning products, for example, are expected not only to cleanse and refresh the human body, but also to have a caring effect. In particular, the skin should not feel dry or taut after cleansing, but smooth, soft, and moisturized.

In the past, therefore, oils were added to detergents to increase the care effect and ways and means were sought to stabilize the oils in the detergents in the long term. In this way, cosmetic cleansing emulsions and shower creams have been developed which ensure an optimal balance between cleansing and care in personal hygiene.

In these versions, polyacrylates were mostly used to stabilize the oils.

However, incompatibilities and low biodegradability have led to efforts to replace polyacrylates in cleaning emulsions or shower creams with other stabilizers without loss of quality.

Cosmetic cleansing emulsions based on washing-active surfactants, pre-gelatinized cross-linked starch derivatives and an oil phase have proposed which leave a smooth and velvety skin feeling during and after application.

However, it was found that the storage stability of these cleaning emulsions was not optimal, especially with temperature fluctuations.

### BRIEF SUMMARY

Cosmetic cleaning products and methods are provided herein. In an embodiment, a cosmetic cleaning product includes—based on the total weight of the cleaning product—
a) from about 5 to about 18% by weight of at least one surfactant,
b) from about 1.5 to about 6% by weight of at least one starch and/or at least one hydrophobically modified starch compound,
c) from about 0.0005 to about 1% by weight of cellulose and/or a non-ionic cellulose ether, and
d) from about 0.01 to about 5% by weight of at least one vegetable oil and/or vegetable butter.

In another embodiment, a cosmetic cleaning product includes—based on the total weight of the cleaning product—
ai) from about 1.5 to about 9% by weight of at least one anionic surfactant and
aii) from about 0.8 to about 7% by weight of at least one amphoteric and/or zwitterionic surfactant,
b) from about 1.5 to about 6% by weight of hydroxypropylated starch phosphate esters,
c) from about 0.0005 to about 1% by weight of cellulose,
di) at least one vegetable oil in a quantity of from about 0.01 to about 5% by weight; and
dii) at least one vegetable butter in an amount of from about 0.01 to about 5% by weight.

In another embodiment, a method includes applying a cosmetic cleaning product as a shower gel and/or shampoo. The cosmetic cleaning products and methods are provided herein. In an embodiment, a cosmetic cleaning product includes—based on the total weight of the cleaning product—
a) from about 5 to about 18% by weight of at least one surfactant,
b) from about 1.5 to about 6% by weight of at least one starch and/or at least one hydrophobically modified starch compound,
c) from about 0.0005 to about 1% by weight of cellulose and/or a non-ionic cellulose ether, and
d) from about 0.01 to about 5% by weight of at least one vegetable oil and/or vegetable butter.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure was therefore based on the task of providing cleansing and caring cosmetic cleansing products with excellent appearance and feel (lotion character), which are extremely mild and leave a feeling of freshness and care on the skin.

In particular, the cleaning products should have excellent long-term and storage stability without the need to add additional stabilizing agents containing polyacrylates.

These tasks were solved by providing a cosmetic cleansing product combining vegetable oils and/or vegetable butters with detergent surfactants and a specific mixture of starch(derivatives) and cellulose(derivatives).

The cleaning products as contemplated herein have the advantage that they do not show any separation or deposits even after major temperature changes, their fragrance and the overall appearance remain unchanged and stable.

Visually and haptically like a body lotion, they still have excellent foaming and cleansing properties (excellent foaming behavior, foam quantities and foam qualities), while maintaining the skin's moisture balance during and after application of the products. The cosmetic cleansing products leave a light, pleasant film on the skin, making it feel as if the skin has been creamed.

A first object of the present disclosure is therefore a cosmetic cleaning product which—based on its total weight—is
a) from about 5 to about 18% by weight of at least one surfactant,
b) from about 1.5 to about 6% by weight of at least one starch and/or at least one hydrophobically modified starch compound,
c) from about 0.0005 to about 1% by weight of cellulose and/or a non-ionic cellulose ether,
d) from about 0.01 to about 5% by weight of at least one vegetable oil and/or vegetable butter.

The cleaning products as contemplated herein are preferably present as liquid compositions, such as wash emulsion, shower cream, shower balm, cream bath, liquid soap and/or shower bath, and preferably contain the active ingredients a) to d) in a suitable cosmetic carrier.

In the context of the present disclosure, this is preferably understood to mean an aqueous carrier which (based on the total weight of the cosmetic cleansing products) preferably contains at least about 65% by weight, particularly preferably at least about 70% by weight and particularly preferably at least about 75% by weight of water.

For further stabilization and/or for further enhancement of the skin care properties—in particular for strengthening the lotion character and smoothing or moisturizing the skin—it may be advantageous if the cleansing products as contemplated herein additionally contain glyceryl ester and/or polyglycerol ester besides the active agents a) to d).

Suitable glycerol esters within the meaning of the present disclosure are preferably glycerol mono- and/or diesters of glycerol with linear or branched, saturated or unsaturated C₈-C₂₄ carboxylic acids.

Examples of particularly suitable glycerol mono- and/or diesters are compounds known under the INCI designations Glyceryl Laurate, Glyceryl Palmitate, Glyceryl Stearate and/or Glyceryl Oleate. Glyceryl Laurate is particularly preferred.

The glycerol esters can be added to the cleaning products as contemplated herein individually or as a mixture, in pure form or (to facilitate incorporation) as a mixture with water and surfactants. Corresponding products are known and available, for example under the names Antil® HS 60, Emulgade® EQ or Lamesoft® PO 65.

Glycerol esters can be used in the cleaning products as contemplated herein (based on their total weight) preferably in quantities of from about 0.05 to about 2% by weight, more preferably of from about 0.1 to about 1.75% by weight, particularly preferably of from about 0.15 to about 1.5% by weight and in particular of from about 0.2 to about 1.25% by weight.

Suitable polyglycerol esters within the meaning of the present disclosure are preferably esters of polyglycerols comprising from about 2 to about 10 glycerol units with linear or branched, saturated or unsaturated C₈-C₂₄ carboxylic acids.

Examples of particularly suitable polyglycerol esters are those listed under the INCI designations Polyglyceryl-2 Caprate, Polyglyceryl-2 Dipolyhydroxystearate, Polyglyceryl-2 Laurate, Polyglyceryl-2 Polyhydroxystearate, Polyglyceryl-3 Caprylate, Polyglyceryl-3 Cocoate, Polyglyceryl-3 Diisostearate, Polyglyceryl-3 Laurate, Polyglyceryl-3 Oleate, Polyglyceryl-3 Palmitate, Polyglyceryl-3 ricinoleates, polyglyceryl-3 stearates, polyglyceryl-4 caprates, polyglyceryl-5 laurates, polyglyceryl-5 oleates, polyglyceryl-5 dioleates, polyglyceryl-6 caprylates, Polyglyceryl-6 dicaprate, polyglyceryl-6 oleate, polyglyceryl-6 stearate, polyglyceryl-3 ricinoleate, polyglyceryl-10 laurate and/or polyglyceryl-10 diisostearate known compounds.

The polyglycerol esters can be added to the cleaning products as contemplated herein individually or as a mixture. Corresponding products are known and available, for example under the names Tego Solve® 61 from Evonik.

Polyglycerol esters can be used in the cleaning products as contemplated herein (based on their total weight) preferably in quantities of from about 0.05 to about 5% by weight, more preferably of from about 0.1 to about 4.5% by weight, particularly preferably of from about 0.15 to about 4% by weight and in particular of from about 0.2 to about 3.5% by weight.

In a first preferred form, the cosmetic cleaning products as contemplated herein contain—based on their total weight—from about 0.05 to about 2% by weight of at least one glyceryl ester and/or from about 0.05 to about 5% by weight of at least one polyglyceryl ester.

Within this version, it is particularly preferred if the cosmetic cleansing products as contemplated herein contain at least one glyceryl monoester, preferably Glyceryl Laurate, Glyceryl Palmitate, Glyceryl Stearate and/or Glyceryl Oleate and especially Glyceryl Laurate.

The cosmetic cleaning products as contemplated herein contain at least one surfactant a).

The at least one surfactant a) should ideally be balanced in terms of mildness (skin compatibility) and foaming properties.

This can be controlled by selecting surfactant quantities and/or surfactant types.

Surfactant totals particularly suitable for achieving a satisfactory cleaning performance are from about 5 to about 18% by weight, more preferably from about 6 to about 17% by weight, particularly preferably from about 7.5 to about 16% by weight and especially from about 8 to about 15% by weight.

Surfactant types are preferably selected from wash-active, mild anionic, amphoteric, zwitterionic and/or non-ionic surfactants.

Particularly preferred cleaning products as contemplated herein contain at least one anionic surfactant ai), which is responsible for the generation of satisfactory foam quantities and foam properties. A mixture of at least one anionic surfactant ai) and at least one mild co-surfactant, which can preferably be selected from amphoteric and/or zwitterionic aii) and/or nonionic surfactants, has been found to achieve an optimum balance between mildness and foaming properties of the cleaning products as contemplated herein.

In a second preferred embodiment, the cleaning products as contemplated herein therefore contain
ai) from about 1.5 to about 9% by weight of at least one anionic surfactant and
aii) from about 0.8 to about 7% by weight of at least one amphoteric and/or zwitterionic surfactant,
   where the quantities indicated refer to the total weight of the cosmetic cleansing product.

Suitable anionic surfactant types ai), which can be used in the cleaning products as contemplated herein, include:
linear and branched fatty acids with from about 8 to about 30 C atoms (Soaps),
Ethercarboxylic acids of the formula R—O—(CH₂—CH₂O)ₓ—CH₂—COOH, in which R is a linear or branched, saturated or unsaturated alkyl group having 8 to 30 C atoms and x=0 or 1 to 16
Acylsarcosides with from about 8 to about 24 C atoms in the acyl group (sarcosinate surfactants)
Acyltaurides with from about 8 to about 24 C atoms in the acyl group (taurate surfactants),
Acyl isethionates with from about 8 to about 24 C atoms in the acyl group (isethionate surfactants),
Sulphosuccinic acid mono- and/or dialkyl esters with from about 8 to about 24 C atoms in the alkyl group and Sulphosuccinic acid mono-alkyl polyoxyethyl esters with from about 8 to about 24 C atoms in the alkyl group and from about 1 to about 6 oxyethyl groups (sulphosuccinate surfactants),
Alpha-olefin sulphonates with from about 8 to about 24 C atoms (alpha-olefin sulphonate surfactants),
Alkyl sulphates and/or alkyl ether sulphate salts of the formula R—(OCH₂—CH₂)ₙ—O—SO₃X, in which R is preferably a straight-chain or branched, saturated or unsaturated alkyl group containing from about 8 to about 30 carbon atoms, x is the number 0 or 1 to 12 and X is an alkali metal, alkaline earth metal, ammonium or alkanolamine ion,
Sulfonates of unsaturated fatty acids with from about 8 to about 24 C atoms and from about 1 to about 6 double bonds,
Esters of tartaric acid and citric acid with alcohols which are addition products of about 2-15 molecules of ethylene oxide and/or propylene oxide to fatty alcohols with from about 8 to about 22 C atoms, and/or
Alkyl and/or alkenyl ether phosphates of the formula

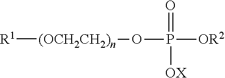

in which R¹ is preferably an aliphatic hydrocarbon radical having from about 8 to about 30 carbon atoms, R² is hydrogen, a radical (CH₂CH₂O)ₙR¹ or X, n is numbers from 0 to about 10 and X is hydrogen, an alkali metal or alkaline earth metal or the group —NR³R⁴R⁵R⁶ where R³ to R⁶ independently of one another are a C₁ bis C₄ hydrocarbon radical.

Alkyl sulphate and/or alkyl ether sulphate salts, (salts of) ether carboxylic acids, isethionates, taurates and/or alpha olefin sulphonates, especially alkyl sulphate and/or alkyl ether sulphate salts, are particularly preferred.

The anionic surfactant(s) ai) is (are) used in the cleaning products as contemplated herein (based on the weight of the total product) preferably in an amount of from about 1.5 to about 9% by weight, more preferably from about 2 to about 8.5% by weight, particularly preferably from about 3 to about 8% by weight, very particularly preferably from about 3.5 to about 7.5% by weight and in particular from about 4 to about 7% by weight.

In a very preferred form, the anionic surfactant ai) is at least one Alkyl sulphate and/or alkyl ether sulphate salt, ether carboxylic acid (salt), isethionate, taurate and/or alpha-olefin sulphonate, in particular an alkyl sulphate and/or alkyl ether sulphate salt, in the above-mentioned quantities used in the cleaning products as contemplated herein.

Suitable amphoteric and/or zwitterionic surfactant types aii) which can be used in the cleaning products as contemplated herein include, for example, one or more compounds of the following formulae (i) to (vii). In which the radical R preferably represents a straight-chain or branched, saturated or mono- or polyunsaturated alkyl or alkenyl radical having from about 7 to about 23 carbon atoms (formulae (i) and (ii)) or a straight-chain or branched, saturated or mono- or polyunsaturated alkyl or alkenyl radical having from about 8 to about 24 carbon atoms (formulae (iii) to (vii)):

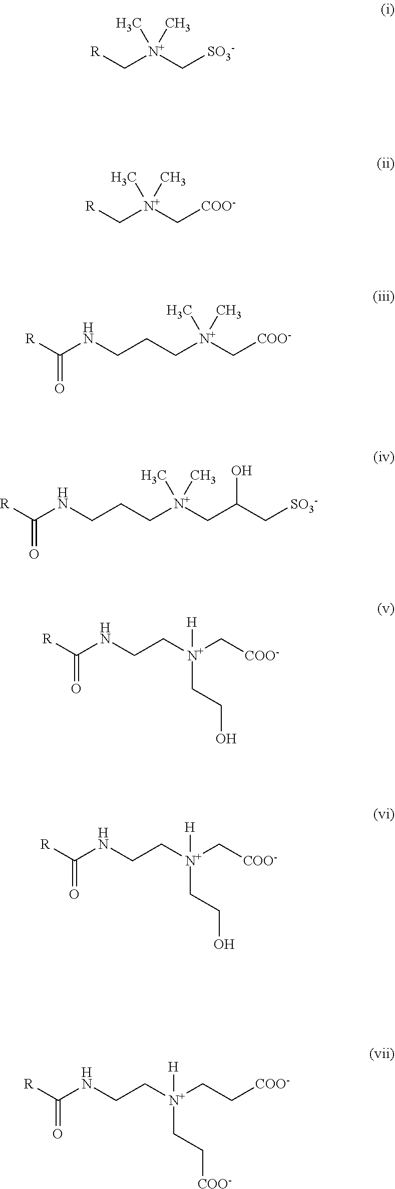

Preferred amphoteric surfactants of the formulae (i) to (vii) mentioned above contain as residue R predominantly a straight-chain or branched, saturated, mono- or polyunsaturated alkyl residue with 8 to 20, more preferably from 8 to 18 and in particular with 8 to 16 C atoms.

Particularly preferred are amphoteric surfactants b), where the rest R is derived from coconut oil.

Particularly preferred are those under the INCI designations Sodium Cocoamphoacetate, Disodium Cocoamphodiacetate, Sodium Lauroamphoacetate, Sodium Lauroamphodiacetate, Sodium Cocoamphopropionate, Disodium Cocoamphodipropionate, Coco Betaine, Lauryl Betaine Cocamidopropylbetaine and/or Lauramidopropylbetaine known and commercially available amphoteric surfactants from several suppliers.

Surfactants with the INCI designations cocamidopropyl betaine, lauramidopropyl betaine, cocoampho(di)acetates and/or lauroapho(di)acetates are particularly preferred.

The amphoteric and/or zwitterionic co-surfactant(s) is (are) used in the cleaning products as contemplated herein (based on their total weight) preferably in an amount of from about 0.8 to about 7% by weight, more preferably from about 1 to about 6.5% by weight, particularly preferably from about 1.5 to about 6% by weight and in particular from about 2 to about 5.5% by weight.

Especially preferred anionic surfactants ai) within the meaning of the present disclosure are alkyl(ether)sulfates due to their excellent foaming properties. These are preferably combined with cocamidopropylbetaine and/or cocoampho(di)acetate as co-surfactant aii) to increase mildness. A combination of alkyl (ether) sulphates, cocamidopropyl betaine and cocoampho(di)acetates is particularly preferred.

In order to further increase the mildness and/or foam properties of the cleaning products as contemplated herein, it may be advantageous in a further preferred version if the compositions additionally contain—based on their weight—from about 0.5 to about 5% by weight, particularly preferably from about 0.6 to about 4% by weight and in particular from about 0.7 to about 3% by weight of at least one nonionic surfactant aiii).

Particularly preferred non-ionic surfactants aiii) are
Fatty acid alkanolamides, preferably C₈-C₂₄-carboxylic acid mono- and/or di-(C₂-C₄)-alkanolamides and especially the compounds known under the INCI designations Cocamide MEA and/or Cocamide MIPA, alkyl polyglucosides, preferably the compounds known under the INCI designations coco glucosides, lauryl glucosides and/or decyl glucosides
C₈-C₃₀ fatty acid mono- and diesters of addition products of from about 1 to about 30 moles of ethylene oxide to glycerol, especially the compound known under the INCI designation PEG-7 Glyceryl Cocoate,
C₅-C₃₀ fatty acid mono- and diesters of glycerol, in particular the compound known under the INCI name Glyceryl Caprylate/Caprate, glucamides, the compounds known under the INCI designations cocoyl methyl glucamides or lauroyl/myristoyl methyl glucamides.
   Alkyl polyglucosides are particularly preferred because of their foam-stabilizing properties.

The cosmetic cleaning products as contemplated herein contain at least one starch and/or at least one hydrophobically modified starch compound b).

Suitable starches within the meaning of the present disclosure are preferably starches obtained from potatoes, maize, rice, peas, acorns, chestnuts, barley, wheat, bananas, sago, millet, sorghum, oats, barley, rye, beans, batata, maranta or manioc Especially preferred are tapioca starch, potato starch, corn starch and/or rice starch.

Hydrophobically modified starch compounds within the meaning of the present disclosure are preferably starch compounds from corn, wheat, rice, tapioca, potatoes and/or sago which have been hydrophobically modified.

For the purposes of this present disclosure, “hydrophobic modification” means chemical cross-linking, for example by ionic cross-linking, with calcium, aluminum and/or polyphosphates—preferably with polyphosphates—and/or chemical modification by introducing hydrophobic groups.

The term “hydrophobic groups” preferably refers to non-ionic residues such as hydroxyalkyl groups, whereas “hydroxyalkyl groups” preferably means hydroxyethyl, hydroxypropyl and/or hydroxybutyl groups.

Particularly preferred hydrophobically modified starch compounds within the meaning of the present disclosure are crosslinked and carry hydrophobic, non-ionic substituents, particularly preferably hydroxypropyl groups.

The hydrophobically modified starch compounds known under the INCI designations Hydroxyethyl Starch Phosphate and Hydroxypropyl Starch Phosphate are particularly preferred.

Hydroxypropyl Starch Phosphate is particularly preferred.

Corresponding products are known and available, for example under the names Structure® XL, Structure® ZEA or Stardesign® Care.

Starch and/or hydrophobically modified starch compounds b) can be used in the cleaning products as contemplated herein (based on their total weight) preferably in amounts of from about 1.5 to about 6% by weight, more preferably of from about 1.5 to about 5.5% by weight, particularly preferably of from about 2 to about 5% by weight and especially of from about 2 to about 4.5% by weight.

In a third preferred version, the cleaning products as contemplated herein contain hydroxypropylated starch phosphate esters as hydrophobically modified starch compound in the above-mentioned quantities, preferably a compound known under the INCI designation Hydroxypropyl Starch Phosphates.

The cosmetic cleaning products as contemplated herein contain cellulose and/or a non-ionic cellulose ether c). It was found that even a small amount of component c) significantly improves the stability of cosmetic cleansing products.

Without the addition of compounds c), signs of separation were already visible after a short period of storage. Storage at higher temperatures (>50° C.) also led to discoloration and loss of viscosity.

“Cellulose” in the sense of the present disclosure is preferably understood to mean cellulose obtained by fermentation (fermentation derived cellulose—PDC). FDC can be produced from natural sources—for example from corn syrup—by bacteria. A suitable bacterium for this is, for example, Gluconacetobacter xylinus.

PDC is pure cellulose and chemically identical to cellulose obtained from plants in the usual way. However, PDC fibers have a three-dimensional, net-like structure and a smaller diameter than plant-derived cellulose fibers, which also gives them a higher surface-to-weight ratio. This structure enables particularly good suspension of components, even when FDC is used in small quantities.

“Nonionic cellulose ethers” within the meaning of the present disclosure can preferably be selected from the group of methyl cellulose, ethyl cellulose, propyl cellulose, methylethyl cellulose, carboxymethyl cellulose, ethylcarboxymethyl cellulose, hydroxyethyl cellulose, hydroxypropyl cellulose, carboxymethylhydroxyethyl cellulose, methylhydroxyethyl cellulose, hydroxypropylmethyl cellulose, ethylhydroxyethyl cellulose, methylethylhydroxyethyl cellulose and mixtures thereof.

Preferably hydroxyethyl cellulose, hydroxypropyl cellulose and/or carboxymethyl cellulose.

Carboxymethyl cellulose is particularly preferred.

Cellulose and/or nonionic cellulose ethers c) can be used in the cleaning products as contemplated herein (based on their total weight) preferably in amounts of from about 0.0005 to about 1% by weight, more preferably of from about 0.001 to about 0.9% by weight, particularly preferably of from about 0.0015 to about 0.8% by weight and in particular of from about 0.002 to about 0.7% by weight.

In a fourth preferred version, the cleaning products as contemplated herein contain PDC in the above-mentioned quantities.

In a fifth preferred form, the cleaning products as contemplated herein contain a mixture of cellulose and non-ionic cellulose ethers in the quantities, particularly preferably a mixture of FDC and carboxymethyl cellulose.

Such mixtures are well known and commercially available, for example as aqueous-alcoholic liquid under the trade name Purllon® R-50.

The cosmetic cleaning products as contemplated herein contain at least one vegetable oil and/or one vegetable butter d).

The presence of oils and/or butters in cosmetic cleansing products as contemplated herein enhances their caring effect and leaves the skin feeling pleasantly soft and smooth.

Suitable vegetable oils within the meaning of the present disclosure are for example coconut oil, (sweet) almond oil, walnut oil, peach kernel oil, apricot kernel oil, avocado oil, tea tree oil, soybean oil, cotton seed oil, sesame oil, sunflower oil, tsubaki oil, evening primrose oil, rice bran oil, palm oil, palm kernel oil, mango kernel oil, Cranberry oil, sea buckthorn oil, lady's smock oil, thistle oil, macadamia nut oil, grape seed oil, amaranth seed oil, argan oil, bamboo oil, olive oil, wheat germ oil, pumpkin seed oil, mallow oil, rose hip seed oil, hazelnut oil, castor oil, safflower oil, corn oil, olive oil, rapeseed oil, canola oil, sasanqua oil, jojoba oil and/or rambutan oil understood.

Sunflower oil, castor oil, cranberry oil and/or rose hip seed oil are particularly preferred.

Suitable vegetable butters within the meaning of the present disclosure are preferably vegetable butters which have a melting range of from about 20 to about 35° C. For example, they can be selected from Shea Butter (INCI designation: Butyrospermum parkii (Shea) Butter), Mango Butter (INCI designation: Mangifera indica (Mango) Seed Butter), Murumuru Butter (INCI designation: Astrocaryum Murumuru Seed Butter), cocoa butter (INCI designation: Theobroma cacao (Cocoa) Seed Butter) and/or Cupuacu Butter (INCI designation: Theobroma grandiflorum Seed Butter).

Shea butter (INCI designation) is particularly preferred: Butyrospermum parkii (Shea) Butter).

Vegetable oils and/or vegetable butters d) can be used in the cleaning products as contemplated herein (based on their total weight) preferably in amounts of from about 0.01 to about 5% by weight, more preferably of from about 0.03 to about 4% by weight, particularly preferably of from about 0.05 to about 3% by weight and in particular of from about 0.1 to about 2.5% by weight.

In a sixth preferred form, the cleaning products as contemplated herein contain (based on their total weight)
di) at least one vegetable oil in a quantity of from about 0.01 to about 5% by weight; and
dii) at least one vegetable butter in an amount of from about 0.01 to about 5% by weight.

To further support the stability, the viscosity, to optimize the flow behavior as well as to enhance the lotion character, it can be advantageous if the cleaning products as contemplated herein contain an additional thickening agent—preferably xanthan gum (xanthan gum).

In another preferred version, the cleaning products as contemplated herein additionally contain from about 0.05 to about 2% by weight of at least one xanthan gum (xanthan gum).

Xanthan gum is a natural, renewable raw material and is excreted as an anionic polysaccharide by the bacterium Xanthomonas campestris.

The molecular weight of the xanthan gum used is preferably from about 2×10⁶ to about 20×10⁶ g/mol.

As molecular building blocks, xanthan gum contains D-glucose, D-mannose, D-glucuronic acid, acetate, and pyruvate in any molar ratio of from about 28 to about 30 to about 20 to about 17 to about 5.1 to about 6.3. The polymer backbone of xanthan gum is formed from a cellulose chain of β-1.4-bound glucose units. Xanthan contains structural units of the following formula

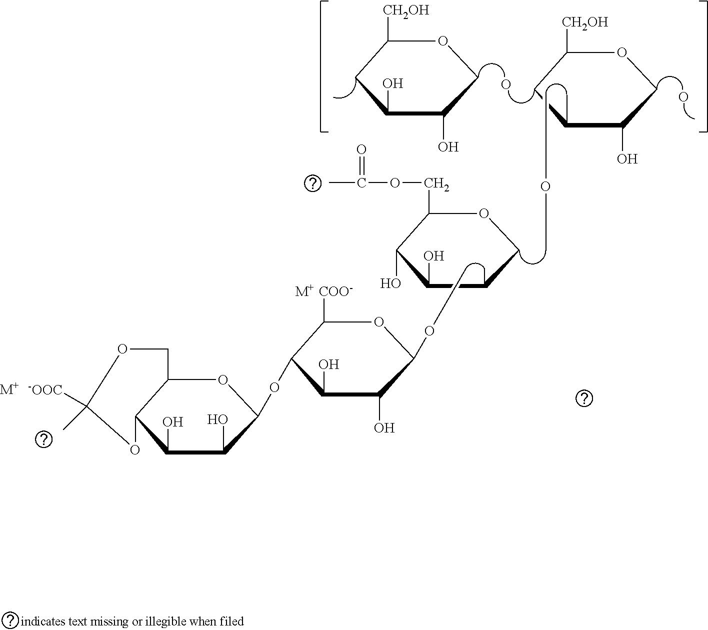

Heat-treated xanthan gum can also be used in the cosmetic cleansing products as contemplated herein.

As contemplated herein, heat-treated Xanthan gum is Xanthan gum that is exposed to heat of at least 40° C. The resulting heat-treated xanthan gum has improved dispersibility and can be dispersed in water faster than xanthan gum that has not been subjected to heat treatment. The preferably suitable heat-treated Xanthan gum has a viscosity of at least from about 25000 to about 40000 mPa·s in a about 1% by weight aqueous solution (Brookfield DV-I viscometer, spindle #6 at 23° C. and 10 rpm). Preferably used heat-treated Xanthan gums provide a pH of from about 4.0 to about 6.0 at 23° C. when prepared in a 1% by weight aqueous solution.

Preferably, Xanthan gum—regardless of whether it is heat-treated or non-heat-treated or a mixture of both—is used in narrower quantity ranges. Here, preferred cleaning products as contemplated herein contain from about 0.1 to about 1.75% by weight, preferably from about 0.15 to about 1.5% by weight, more preferably from about 0.15 to about 1.25% by weight and in particular from about 0.2 to about 1% by weight of xanthan gum.

In another preferred form, the cleaning products as contemplated herein additionally contain from about 1 to about 10% by weight of at least one polyol.

Suitable polyols within the meaning of the present disclosure are preferably polyols which contain from about 2 to about 10 carbon atoms, preferably from about 3 to about 8 carbon atoms, and from about 2 to about 8 hydroxyl groups, preferably from about 2 to about 6 hydroxyl groups, in their molecule.

Examples of preferred polyols are: Ethylene glycol, 1,2-propylene glycol, 1,3-propylene glycol, dipropylene glycol, 1,3-butylene glycol, 1,4-butylene glycol, isoprene glycol, 1,2-pentanediol, 1,3-pentanediol, 1,2-hexanediol, 1,6-hexanediol, glycerol, diglycerol, sorbitol or mixtures of these polyols.

Water-soluble polyols are particularly preferred.

Particularly preferred are 1,2-propylene glycol, glycerol, 1,3-butylene glycol, 1,6-hexanediol, diglycerol and/or sorbitol; especially preferred are glycerol and sorbitol.

The proportion by weight of the polyol, in particular of glycerol and/or sorbitol, in the total weight of the cleaning products as contemplated herein is preferably from about 1.5 to about 9% by weight, particularly preferably from about 2 to about 8% by weight and in particular from about 2.5 to about 7% by weight.

By combining the above-mentioned essential (a)-d)) and optional ingredients, it is possible to provide particularly stable cleansing products which are visually and haptically like a body lotion but still have excellent foaming and cleansing properties.

It is not necessary to add other synthetic stabilizing products to the cleaning products. In particular, the use of polyacrylate polymers and their derivatives is not necessary.

In another preferred design the cleaning products as contemplated herein are essentially free of polymeric stabilizing agents.

Within this version, it is particularly preferred if the cleaning products as contemplated herein are essentially free from polymeric stabilizing agents based on acrylic acid and/or acrylic acid derivatives.

As contemplated herein, “substantially free” is understood to mean that the cleaning products contain—based on their total weight—a maximum of about 0.01% by weight, preferably a maximum of about 0.005% by weight and in particular 0% by weight of polymeric stabilizing agents, in particular polymeric stabilizing agents based on acrylic acid and/or acrylic acid derivatives.

In addition to the essential and optional ingredients, the cosmetic cleansing products as contemplated herein may contain at least one further conditioning active ingredient, preferably a skin conditioning active ingredient from the group of cationic polymers, plant extracts, allantoin and/or vitamins, to further enhance their skin care properties.

Suitable cationic polymers are for example:
quaternized cellulose derivatives, as referred to under the names Celquat® and Polymer JR® are available on the market,
hydrophobically modified cellulose derivatives, such as the cationic polymers sold under the trade name SoftCat®,
cationic alkyl polyglycosides,
cationized honey, for example the commercial product Honeyquat® 50,
cationic guar derivatives, in particular the products marketed under the trade names Cosmedia Guar N-Hance® and Jaguar®,
polymeric dimethyldiallylammonium salts and copolymers thereof with esters and amides of acrylic acid and methacrylic acid. The products marketed under the names Merquat®100 (poly(dimethyldiallylammonium chloride)) and Merquat®550 (dimethyldiallylammonium chloride-acrylamide copolymer) are examples of such cationic polymers,
Copolymers of vinylpyrrolidone with quaternized derivatives of dialkylaminoalkyl acrylate and methacrylate, such as vinylpyrrolidone-dimethylaminoethyl methacrylate copolymers quaternized with diethyl sulfate. Such compounds are commercially available under the names Gafquat® 734 and Gafquat® 755,

Vinylpyrrolidone-vinylimidazolium methochloride copolymers, as offered under the names Luviquat® FC 370, FC 550, FC 905, and HM 552,
quaternized polyvinyl alcohol, and the products marketed under the designations
Polyquaternium 2, Polyquaternium 17, Polyquaternium 18, Polyquaternium-24, Polyquaternium 27, Polyquaternium-32, Polyquaternium-37, Polyquaternium 74 and Polyquaternium 89 known polymers.

Preferred are cationic polysaccharide polymers such as quaternized cellulose polymers, hydrophobically modified cationic cellulose derivatives and/or cationic guar derivatives and polymeric dimethyldiallylammonium salts and their copolymers with esters and amides of acrylic acid and methacrylic acid. The cationic polymers known under the INCI designations Guar Hydroxypropyltrimonium Chloride, Polyquaternium-10, Polyquaternium-37, Polyquaternium-67 Polyquaternium-72, Polyquaternium-6 and/or Polyquaternium-7 are particularly preferred; Guar Hydroxypropyltrimonium Chloride is particularly preferred.

The cosmetic cleaning products as contemplated herein contain cationic polymers—preferably guar hydroxypropyltrimonium chloride—preferably in a proportion of from about 0.01 to about 2% by weight, particularly preferably from about 0.025 to about 1.5% by weight and especially from about 0.05 to about 1% by weight of the total weight of the cleaning products.

Suitable plant extracts are mainly the extracts of green tea, oak bark, nettle, witch hazel, hops, henna, chamomile, burdock root, horsetail, hawthorn, lime blossom, almond, Aloe Vera, spruce needle, horse chestnut, date palm, cinnamon tree, sandalwood, juniper, coconut, mango, apricot, lime, wheat, kiwi, melon, orange, grapefruit, sage, rosemary, birch, mallow, Valerian, cuckooflower, thyme, lemon balm, yarrow, lemon balm, cowslip, marshmallow, meristem, ginseng, coffee, cocoa, moringa, ginger root and Ayurvedic plant extracts such as Aegle marmelos (Bilva), Cyperus rotundus (Nagar Motha), Emblica officinalis (Amalki), Morida citrifolia (Ashyuka), Tinospora cordifolia (Guduchi), Santalum album, (Chandana), Crocus sativus (Kumkuma), Cinnamonum zeylanicum and Nelumbo nucifera (Kamala), sweet grasses such as wheat, barley, rye, oats, spelt, maize, the various varieties of millet (panicle millet, finger millet, sorghum as examples), sugar cane, ryegrass, meadow foxtail, smooth oats, ostrich grass, meadow fescue, pipe grass, bamboo, cotton grass, lamp-cleaning grass, Andropogonodeae (Imperata Cylindrica also known as flame grass or cogon grass), buffalo grass, silty grasses, dog-toothed grasses, love grasses, Cymbopogon (lemon grass), Oryzeae (rice), Zizania (wild rice) Beach grass, perennial oats, honey grasses, trembling grasses, meadow-grass, couch grass and echinacea, in particular Echinacea angustifolia DC, Echinacea paradoxa (Norton), Echinacea simulata, E. atrorubens, E. tennesiensis, Echinacea strigosa (Mc Gregor), Echinacea laevigata, Echinacea purpurea (L.) Moench and Echinacea pallida (Nutt), all types of vine and pericarp of Litchi chinensis.

Suitable extracts can be obtained from the fruits, seeds, flowers, roots, leaves and/or bark of the above-mentioned plants.

As contemplated herein, the plant extracts can be used both in pure and diluted form. If they are used in diluted form, they usually contain approx. 2-80% by weight of active substance and as solvent the extracting agent or extracting agent mixture used in their extraction. Suitable extraction agents are usually water and/or alcohols.

Aqueous-alcoholic extracts of chamomile are particularly preferred.

The plant extract(s) can be used in the cosmetic cleansing products as contemplated herein, preferably in total amounts of from about 0.001 to about 1% by weight, preferably from about 0.05 to about 0.75% and in particular from about 0.1 to about 0.5% by weight, the amounts being based on the weight of the cosmetic cleansing products.

Allantoin can be added to the cosmetic cleansing products as contemplated herein, preferably to reduce skin dehydration during cleansing and to increase the suppleness and smoothness of the skin, in amounts of from about 0.001 to about 1% by weight, more preferably from about 0.005 to about 0.75% by weight and more particularly from about 0.001 to about 0.5% by weight, the amounts being based on the total weight of the cosmetic cleansing products.

Suitable “vitamins” are preferably the following vitamins, provitamins and vitamin precursors and their derivatives:
Vitamin A: the group of substances known as vitamin A includes retinol (vitamin A₁) and 3,4-didehydroretinol (vitamin A₂). The β-carotene is the provitamin of retinol. Vitamin A components include vitamin A acid and its esters, vitamin A aldehyde and vitamin A alcohol and its esters such as palmitate and acetate.
Vitamin B: the vitamin B group or vitamin B complex includes
Vitamin B₁ (Thiamine)
Vitamin B₂ (Riboflavin)
Vitamin B₃. The compounds nicotinic acid and nicotinamide (niacinamide) are often referred to under this name.
Vitamin B₅ (pantothenic acid and panthenol). Within this group, panthenol is preferred. Usable derivatives of panthenol are the esters and ethers of panthenol, pantolactone and cationically derivatized panthenols. Individual representatives include panthenol triacetate, panthenol monoethyl ether and its monoacetate and cationic panthenol derivatives.
Vitamin B₆ (pyridoxine as well as pyridoxamine and pyridoxal).
Vitamin C (ascorbic acid): use in the form of palmitic acid ester, glucosides or phosphates may be preferred. Use in combination with tocopherols may also be preferred.
Vitamin E (tocopherols, especially α-tocopherol).
Vitamin P the term “vitamin F” usually refers to essential fatty acids, especially linoleic acid, linolenic acid, and arachidonic acid.
Vitamin H: Vitamin H is the compound (3aS,4S,6aR)-2-oxohexahydrothienol[3,4-d]-imidazole-4-valeric acid, for which the trivial name biotin has since become established.
   Especially preferred are vitamins, provitamins and vitamin precursors from the groups A, B, E and H. Nicotinamide, biotin, pantolactone and/or panthenol are particularly preferred; nicotinamide and/or panthenol are especially preferred.

The proportion by weight of the vitamin(s), vitamin derivative(s) and/or vitamin precursor(s) in the total weight of the cosmetic cleansing products as contemplated herein is preferably from about 0.005 to about 2% by weight, more preferably from about 0.006 to about 1.5% by weight, more preferably from about 0.008 to about 1% by weight and more preferably from about 0.01 to about 0.50% by weight.

Other active ingredients, auxiliaries and additives that can be used in the cosmetic cleaning products as contemplated herein are, for example:
UV-Filter,
Structurants like maleic acid and lactic acid,
Dyes to color the product,
Substances for adjusting the pH value, for example α- and β-hydroxycarboxylic acids such as citric acid, lactic acid, malic acid, glycolic acid,
Complexing agents like EDTA, NTA, β-alaninediacetic acid and phosphonic acids,
Ceramide. Ceramides are N-acyl sphingosine (fatty acid amides of sphingosine) or synthetic analogues of such lipids (so-called pseudo-ceramides),
Preservatives such as benzoic acid, salicylic acid and/or their salts,
Blowing agents such as propane-butane mixtures, N₂O, dimethylether, CO₂ and air,
Antioxidants,
Pearlescent agents such as EGDS or PEG-3 distearate,
Opacifiers like the compounds known under the INCI designation styrene/acrylates copolymer,
Perfume components,
Viscosity regulators like electrolyte salts (NaCl).

A second subject-matter of the present disclosure is the cosmetic use of the cosmetic cleaning products as contemplated herein as shower gel and/or shampoo.

### EXAMPLES

The following present disclosure-based cosmetic cleansing products have been manufactured (the quantities given are based on weight %):

| | 1 | 2 | 3 | 4 | 5 | 6 |
|---|---|---|---|---|---|---|
| Sodium Laureth Sulfate | 1.5-9 | 1.5-9 | 1.5-9 | 1.5-9 | 1.5-9 | 1.5-9 |
| Cocamidopropyl betaine | 0.8-7 | | 0.8-7 | 0.8-7 | 0.8-7 | 0.8-7 |
| Disodium | | 0.8-7 | 0.8-7 | 0.8-7 | 0.8-7 | 0.8-7 |
| Cocoamphodiacetate | | | | | | |
| Coco glucosides | 0.5-5 | 0.5-5 | | 0.5-5 | | 0.5-5 |
| Potato starch | | | | 1.5-6 | | |
| Stardesign Care ®¹ | 1.5-6 | 1.5-6 | 1.5-6 | | 1.5-6 | 1.5-6 |
| FDC | | | | 0.0005-1 | | |
| Carboxymethylcellulose | | | | | 0.0005-1 | |
| Purllon R-50 ®² | 0.0005-1 | 0.0005-1 | 0.0005-1 | | 0.0005-1 | 0.0005-1 |
| Vegetable oil (e.g. sunflower | 0.01-5 | | 0.01-5 | 0.01-5 | 0.01-5 | 0.01-5 |
| oil, castor oil and/or rose hip | | | | | | |
| seed oil) | | | | | | |
| Shea Butter | | 0.01-5 | 0.01-5 | 0.01-5 | 0.01-5 | 0.01-5 |
| Antil HS 60 ®³ | 0.05-2 | | | 0.05-2 | 0.05-2 | 0.05-2 |
| Tego Solve 61 ®⁴ | | 0.05-5 | 0.05-5 | | | |
| Chamomile extract | 0.001-1 | 0.001-1 | | 0.001-1 | | 0.001-1 |
| Allantoin | 0.001-1 | 0.001-1 | 0.001-1 | | 0.001-1 | 0.001-1 |
| Perfume | 0.1-2 | 0.1-2 | 0.1-2 | 0.1-2 | 0.1-2 | 0.1-2 |
| Guar Hydroxypropyltrimonium | 0.01-2 | 0.01-2 | | 0.01-2 | | 0.01-2 |
| Chloride | | | | | | |
| Preservatives | q.s. | q.s. | q.s. | q.s. | q.s. | q.s. |
| pH adjusting agent (up to pH | q.s. | q.s. | q.s. | q.s. | q.s. | q.s. |
| 4.5-5.5) | | | | | | |
| Water | ad 100 | ad 100 | ad 100 | ad 100 | ad 100 | ad 100 |

The following commercial products were used:
^{®1} INCI designation: Hydroxypropyl Starch Phosphate; Cargill
^{®2} INCI designation: Water, Glycerin, FDC, Sodium Carboxymethyl Cellulose; Kelco
^{®3} INCI designation: Cocamidopropyl betaine, Glyceryl laurate; Evonik
^{®4} INCI designation: Polyglyceryl-6 caprylate, Polyglyceryl-3 cocoate, Polyglyceryl-4 caprate, Polyglyceryl-6 rizinoleate; Evonik

The cosmetic cleansing compositions of examples 1-6 as contemplated herein show an improved stability and texture compared to comparative formulations (in which one or more active ingredients a) to d) are missing). They have a long-lasting effect on rough and/or scaly skin and give it a smooth, soft feel that can be felt for a long time after rinsing the compositions.

In particular, the addition of cellulose (derivatives)n in addition to starch and/or hydrophobically modified starch compounds as stabilizing agents can prevent separations and/or deposits as well as changes in the fragrance or overall appearance. The addition of further stabilizing agents (especially stabilizing agents containing polyacrylates) is not necessary.

Furthermore, the cleaning products as contemplated herein show excellent foaming behavior as well as good foam quantities and foam qualities.

The cosmetic cleansing products also leave a light, pleasant film on the skin, making it feel as if the skin has been creamed.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. Cosmetic cleaning product comprising—based on the total weight of the cleaning product—
d) from about 5 to about 18% by weight of at least one surfactant,
e) from about 1.5 to about 6% by weight of at least one starch and/or at least one hydrophobically modified starch compound,
f) from about 0.0005 to about 1% by weight of cellulose and/or a non-ionic cellulose ether,
g) from about 0.01 to about 5% by weight of at least one vegetable oil and/or vegetable butter.

2. Cosmetic cleansing product according to claim 1, additionally comprising—based on its total weight—from about 0.05 to about 2% by weight of at least one glyceryl ester and/or from about 0.05 to about 5% by weight of at least one polyglyceryl ester.

3. Cosmetic cleansing product according to claim 2, comprising at least one glyceryl monoester.

4. Cosmetic cleansing product according to claim 1, comprising as surfactant a) a mixture of
ai) from about 1.5 to about 9% by weight of at least one anionic surfactant and
aii) from about 0.8 to about 7% by weight of at least one amphoteric and/or zwitterionic surfactant,
where the quantities indicated refer to the total weight of the cosmetic cleansing product.

5. Cosmetic cleansing product according to claim 1, comprising hydroxypropylated starch phosphate esters as hydrophobically modified starch compound.

6. Cosmetic cleansing product according to claim 1, comprising as component c) cellulose.

7. Cosmetic cleaning product according to claim 1, comprising
di) at least one vegetable oil in a quantity of from about 0.01 to about 5% by weight; and
dii) at least one vegetable butter in an amount of from about 0.01 to about 5% by weight,
where the quantities indicated refer to the total weight of the cosmetic cleansing product.

8. Cosmetic cleaning product according to claim 1, further comprising—based on its total weight—
h) from about 0.05 to about 2% by weight of xanthan gum and/or
i) from about 1 to about 10% by weight of at least one polyol.

9. Cosmetic cleansing product according to claim 1, substantially free of polymeric stabilizing agents.

10. A method comprising applying the cosmetic cleaning product according to claim 1 as a shower gel and/or shampoo.

11. Cosmetic cleansing product according to claim 2, comprising at least one of Glyceryl Laurate, Glyceryl Palmitate, Glyceryl Stearate and/or Glyceryl Oleate.

12. Cosmetic cleansing product according to claim 2, comprising Glyceryl Laurate.

13. Cosmetic cleansing product according to claim 1, comprising a compound known under the INCI designation Hydroxypropyl Starch Phosphates as the hydrophobically modified starch compound.

14. Cosmetic cleansing product according to claim 1, comprising as component c) FDC.

15. Cosmetic cleansing product according to claim 1, comprising as component c) a mixture of cellulose and a non-ionic cellulose ether.

16. Cosmetic cleansing product according to claim 1, comprising as component c) a mixture of FDC and carboxymethyl cellulose.

17. Cosmetic cleansing product according to claim 1, substantially free of polymeric stabilizing agents based on acrylic acid and/or acrylic acid derivatives.

18. Cosmetic cleaning product comprising—based on the total weight of the cleaning product—
ai) from about 1.5 to about 9% by weight of at least one anionic surfactant and
aii) from about 0.8 to about 7% by weight of at least one amphoteric and/or zwitterionic surfactant,
b) from about 1.5 to about 6% by weight of hydroxypropylated starch phosphate esters,
c) from about 0.0005 to about 1% by weight of cellulose,
di) at least one vegetable oil in a quantity of from about 0.01 to about 5% by weight; and
dii) at least one vegetable butter in an amount of from about 0.01 to about 5% by weight.

19. Cosmetic cleansing product according to claim 18, additionally comprising—based on its total weight—from about 0.05 to about 2% by weight of at least one glyceryl ester and/or from about 0.05 to about 5% by weight of at least one polyglyceryl ester.

20. Cosmetic cleansing product according to claim 19, comprising at least one glyceryl monoester.
